(19) United States
(12) Patent Application Publication
(10) Pub. No.: US 2025/0102483 A1
(43) Pub. Date: Mar. 27, 2025

(54) Probe Housing and Probe Device Having a Sensor and a Probe Housing

(71) Applicant: Deutsches Zentrum für Luft- und Raumfahrt e.V., Bonn (DE)

(72) Inventors: Martin Müller, Köln (DE); Manfred Beversdorff, Köln (DE); Guido Stockhausen, Köln (DE)

(21) Appl. No.: 18/578,965

(22) PCT Filed: Jul. 8, 2022
(86) PCT No.: PCT/EP2022/069143

§ 371 Date: Jan. 12, 2024

(30) Foreign Application Priority Data
Jul. 15, 2021 (DE) 10 2021 118 359.6

(51) Int. Cl. G01N 33/00; G01N 21/31

(52) U.S. Cl. CPC G01N 33/0029; G01N 21/31

(57) ABSTRACT

The invention relates to a probe housing for accommodating sensors, including a plurality of coolant conduits distributed in the circumferential direction; at least one flushing medium conduit; and a sensor lead-through hole. The sensor lead-through hole extends at least partially parallel to the at least one flushing medium conduit; and a sensor receptacle into which the sensor lead-through hole opens. The sensor receptacle has a measuring section opening, and the sensor receptacle has a flushing medium outlet conduit having a flushing medium outlet which is connected to the at least one flushing medium conduit, and the flushing medium outlet conduit has a tapering cross-section to form a nozzle shape.

### CROSS-REFERENCE TO RELATED APPLICATIONS

This application is the United States national phase of International Application No. PCT/EP2022/069143 filed Jul. 8, 2022, and claims priority to German Patent Application No. 10 2021 118 359.6 filed Jul. 15, 2021, the disclosures of which are hereby incorporated by reference in their entireties.

### BACKGROUND OF THE INVENTION

### Field of the Invention

The present invention relates to a probe housing and a probe device having a sensor and a probe housing.

### Description of Related Art

For the diagnostics (setting an operating point, system control, troubleshooting, control etc.) of hot gas installations, such as, for example, gas turbines, combustion engines, steam power plants or the like, sensors in probes are used which are guided directly into the hot gas environment or to the edge thereof in a minimally invasive manner. The probe bodies comprise cooling devices via which the probe bodies and the sensor technology housed therein can be cooled.

From DE 10 2012 216 267 A1 such a probe body is known which is formed by a probe body with cooling conduits, wherein a probe head is exchangeable so as to adapt the system to different sensors. Since the probe head has to be cooled, a complex coupling structure is required to lead the cooling medium into the probe head. Further, it is provided for the known probe that a flushing medium can be used to clean the sensor exit face of the probe.

The probe has a plurality of conduits uniformly distributed in the circumferential direction. Some of these conduits are used as cooling conduits, while other conduits serve to supply flushing air.

Due to the design having an exchangeable probe head, the known structure is relatively large and intricate or complex and, as such, susceptible to leakage. However, it is one objective when measuring hot gas installations to affect a hot gas flow as little as possible in order to obtain measuring results of the highest quality possible. Therefore, it is desirable to design a probe with the smallest dimensions possible.

Moreover, in the known probe, the flushing air supplied to the sensor outlet may cause interferences with the hot gas environment. In addition, in prior art, flushing is performed concentrically, whereby increased contamination may occur at the sensor outlet due to vortex formation.

Since the known probe does not use all conduits distributed in the circumference for a cooling function, an irregular cooling of the probe and thus of the sensor may occur in addition.

As a matter of fact, the probe head of the known probe is permanently exposed to the hot gas, so that, even at times when no measurement is performed, the components are under thermal stress and the contamination process can proceed. Therefore, such a structural design requires increased cooling and flushing.

### SUMMARY OF THE INVENTION

Therefore, it is an object of the present invention to provide a probe housing of the type mentioned above, which has a compact design and, in addition, preferably overcomes at least some of the disadvantages mentioned. Further, it is an object of the present invention to provide a probe device having such a probe housing.

The probe housing according to the invention is defined by the features as described herein. The probe device according to the invention is defined by the features as described herein.

The probe housing according to the invention for receiving sensors has a plurality of circumferentially distributed coolant conduits, at least one flushing medium conduit and a sensor lead-through hole. The sensor lead-through hole extends at least partly parallel to the at least one flushing medium conduit. The probe housing further comprises a sensor receptacle, into which the sensor lead-through hole opens, the sensor receptacle comprising a measuring section opening. The sensor receptacle comprises a flushing medium outlet line with a flushing medium outlet connected to the at least one flushing medium conduit. The invention is characterized in that the flushing medium outlet line has a tapering cross section to form a nozzle shape.

The measuring section opening communicates the sensor receptacle with the environment. The sensor receptacle serves to receive a sensor head of a sensor, wherein a measuring outlet of the sensor head can be arranged in the direction of the measuring section opening so that the measurement can be performed through the measuring section opening of the sensor receptacle.

The sensor lead-through hole may serve for fitting the sensor head into the sensor receptacle, for example, wherein, further, the signal lines of the sensor can be passed through the sensor lead-through hole.

The flushing medium serves to protect the measuring section opening or a sensor head inserted in the sensor receptacle. By designing the flushing medium outlet line with a tapering cross section to form a nozzle shape, the flow of the flushing medium can be accelerated. Thereby, it is possible to achieve a particularly effective protection against hot gas flow and a particularly good heat dissipation by the flushing medium. The increased velocity of the flushing medium further improves the flushing performance and reduces the tendency for contamination, since particles are deflected earlier by the flushing medium flow.

It is preferably provided that the sensor lead-through hole and the at least one flushing medium conduit are surrounded by the coolant conduits and are arranged offset from a centre axis of the probe housing. The probe housing according to the invention thus provides that the at least one flushing medium conduit and the sensor lead-through hole are arranged in the interior of the probe housing, wherein the coolant conduits are arranged farther outward and surrounding the at least one flushing medium conduit and the sensor lead-through hole. Due to the arrangement of the sensor lead-through hole and the at least one flushing medium conduit offset from the center axis, these can be arranged in the probe housing in an advantageous and spacesaving manner.

Preferably, it is provided that the coolant conduits are regularly distributed in the circumferential direction. In this manner, a particularly advantageous and uniform cooling of the probe housing is possible. Generally, it may be provided that some of the coolant conduits serves to supply the coolant and the other coolant conduits serve to remove the heated coolant.

Preferably, it is provided that the probe housing is formed by a tubular outer body and an inner body, the inner body being surrounded by the outer body, and the sensor lead-through hole and the at least one flushing medium conduit being arranged in the inner body. The probe housing may also be a monolithic body in which the inner body and the outer body are combined into a single component in the manufacturing process (e.g. by sintering, casting or printing).

Here, it may be provided that the tubular outer body has an oval or elliptic cross section. Thereby, the flow resistance of the probe housing with respect to the hot gas flow may be reduced. The inner body may be adapted to the shape of the outer body.

An arrangement of the sensor lead-through hole and the at least one flushing medium conduit offset from the center axis of the probe housing allows for a design of the probe housing with an outer body having an oval or an elliptical cross section and thus a design with a reduced flow resistance in a particularly advantageous manner.

In one embodiment of the invention, it may be provided that the cross section of the sensor lead-through hole has a shape adapted to a sensor head and/or a sensor.

Designing the invention with a tubular outer body having an oval or elliptical cross section also is of an independent inventive importance and may thus be realized independent of the design of the flushing medium outlet line. Here, the embodiment of the invention with a tubular outer body having an oval or elliptical cross section can be combined with individual, a plurality or all features described above or hereinafter.

It may be provided that the coolant conduits are arranged at the circumference of the inner body. Preferably, it is provided that the coolant conduits are formed by recesses in the inner body, the outer body limiting the coolant conduits on at least one side. Such a structure has proven particularly advantageous. On the one hand, the cooling conduits can advantageously be provided in the inner body in the form of grooves, in which case the cooling conduits are completely closed in the circumferential direction by insertion of the inner body into the outer body. In addition, due to the two-part design, the inner body can be made from another material than the outer body, so that, for example, a particularly heat-resistant material can be used for the outer body.

The coolant conduits may have cross sections that differ in size. Thus, the cooling performance of the individual coolant conduits is adapted to the thermal load on the probe housing. For example, a coolant conduit in a region of the probe housing which, in use, faces a hot gas flow can have a larger cross section than a coolant conduit in a region of the probe housing which, in use, faces away from the hot gas flow.

Preferably, it is provided that an aperture and/or an optically transmissive disc is/ore arranged in or at the measuring section opening, comprising a major surface. Depending on the type of sensor used, the probe housing can thus be adapted to the senso. The aperture and/or the optically transmissive disc may also serve as an additional protection for a sensor head inserted in the sensor receptacle.

Here, it may be provided that the flushing medium outlet is arranged under an acute angle α with respect to the plane in which the major surface is located. Thereby, the flushing medium flows in a direction onto the major surface and not parallel the major surface or away therefrom. Due to the incident flow on the major surface of the disc or aperture, a particularly good heat dissipation can be achieved, since it is ensured that the flushing medium flows across the major surface and thus contacts the same. In addition, the major surface can be cleaned by the flushing medium flushing away particles adhering to the major surface. The angle α may e.g. be between 1.5° to 5°, preferably 2°. In the context of the invention, a flushing medium at an acute angle α with respect to a plane should be understood such that the angle of a center line of the section of the flushing medium outlet extends under the corresponding angle relative to the plane.

The design of the invention with a flushing medium outlet at an acute angle α with respect to a plane in which the major surface is located also is of independent inventive importance and may thus also be realized independently of the design of the flushing medium outlet line with a tapering cross section. Here, the embodiment of the invention with a flushing medium outlet at an acute angle α with respect to a plane in which the major surface is located, can be combined with individual, a plurality or all features described above or hereinafter.

In a particularly preferred embodiment of the invention, it is provided that the flushing medium outlet line is formed as a Laval nozzle. Thereby, the flushing medium can be accelerated to very high velocities which are even higher than the velocity of sound. Thereby, it is possible to achieve a particularly effective protection against hot gas flow and a particularly good heat dissipation by the flushing medium. The flushing medium outlet line or the flushing medium outlet may generally also be arranged parallel to a plane in which the major surface is located, so that the flushing medium flows parallel to the major surface.

Preferably, it is provided that the flushing medium outlet has a rectangular cross section with a long side and a short side, and the flushing medium outlet is arranged with the long side parallel to the major surface of the measuring section. In this way, a flushing medium flow can be generated that is relatively wide and flows across, and thus flushes, a large area of the measuring section.

Preferably, it is provided that the flushing medium outlet has a dimension in the direction of the longitudinal side that is equal or greater than the maximum dimension of the measuring section opening in that direction. Thereby, it is achieved that the flushing medium flow formed upon exiting the flushing medium outlet has a width equal to or larger than the maximum dimension of the measuring section opening in this width direction, so that the flushing medium flow flows across the entire measuring section.

The opening plane of the measuring section opening or the major surface of the aperture arranged in the measuring section opening or of the optically transmissive disc may be arranged orthogonally or in parallel to the center axis. In other words: Due to the orthogonal arrangement with respect to the center axis, it is possible to perform a measurement in the axial direction of the probe. Due to the parallel arrangement of the center axis, it is possible to perform a measurement in the direction orthogonal to the center axis.

Preferably, it is provided that the optically transmissive disc includes an optical element. The optical element may, for example, have a converging property for optical radiation. For example, the optical element may be a lens. The optical element may, for example, be integrated in the optically transmissive disc or be provided in addition to the same.

In the context of the invention, an optically transmissive disc is understood as a disc which, when optical sensors are used, transmits optical radiation that is sufficient for the optical sensor and is in a wavelength range in which the optical sensor operates. For example, the optically transmissive disc can transmit at least 50% of the radiation in the corresponding wavelength range.

In one embodiment of the invention, it is provided that the flushing medium outlet line intersects the measuring section opening, wherein a sealing air flow can be generated in the measuring section opening. In one embodiment of the invention without optically transmissive disc, a sealing air flow is formed using the flushing medium, which provides an effective protection of a sensor head fitted in the sensor receptacle against a hot gas flow. Due to the acceleration of the flow of the flushing medium, the sealing air flow can be generated in a particularly advantageous manner because of the tapering cross section of the flushing medium outlet line.

An embodiment of the invention, in which the flushing medium outlet line intersects the measuring section opening, while a sealing air flow can be generated in the measuring section opening, also is of independent inventive importance and may thus be realized independently of the design of the flushing medium outlet line with a tapering cross section. Here, the embodiment of the invention, in which the flushing medium outlet line intersects the measuring section opening an be combined with individual, a plurality or all features described above or hereinafter.

The invention further provides a probe device with a sensor having a sensor head and a probe housing according to the invention, the sensor head being arranged in the sensor receptacle and a measuring outlet of the sensor head is arranged facing to the measuring section opening. In the probe device, it can be provided that measuring lines leading to the sensor head are arranged passing through the sensor lead-through hole.

### BRIEF DESCRIPTION OF THE DRAWINGS

The terms Fig., Figs., Figure, and Figures are used interchangeably in the specification to refer to the corresponding figures in the drawings.

In the following, embodiments of the invention are described in more detail with reference to the following figures.

In the drawings:

FIG. 1 is a schematic perspective view of a first embodiment of a probe housing according to the invention for receiving a sensor,

FIG. 2 is a schematic sectional view of the probe housing in FIG. 1,

FIG. 3 is a schematic sectional view of the probe housing in FIG. 1 with a first variant of the flushing medium conduit,

FIG. 4 is a schematic sectional view of the probe housing in FIG. 1 with a second variant of the flushing medium conduit,

FIG. 5 is a schematic perspective view of a second embodiment of a probe housing according to the invention for receiving a sensor

### DESCRIPTION OF THE INVENTION

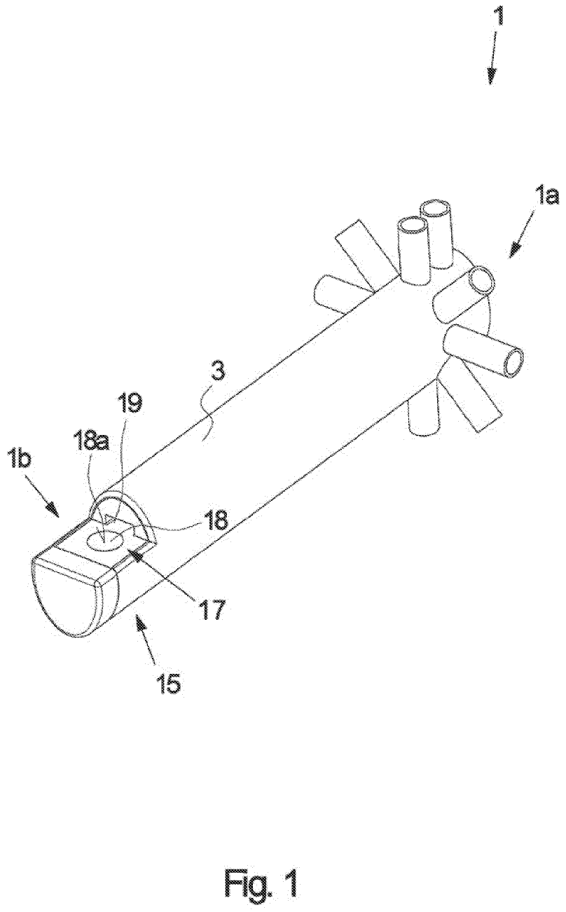

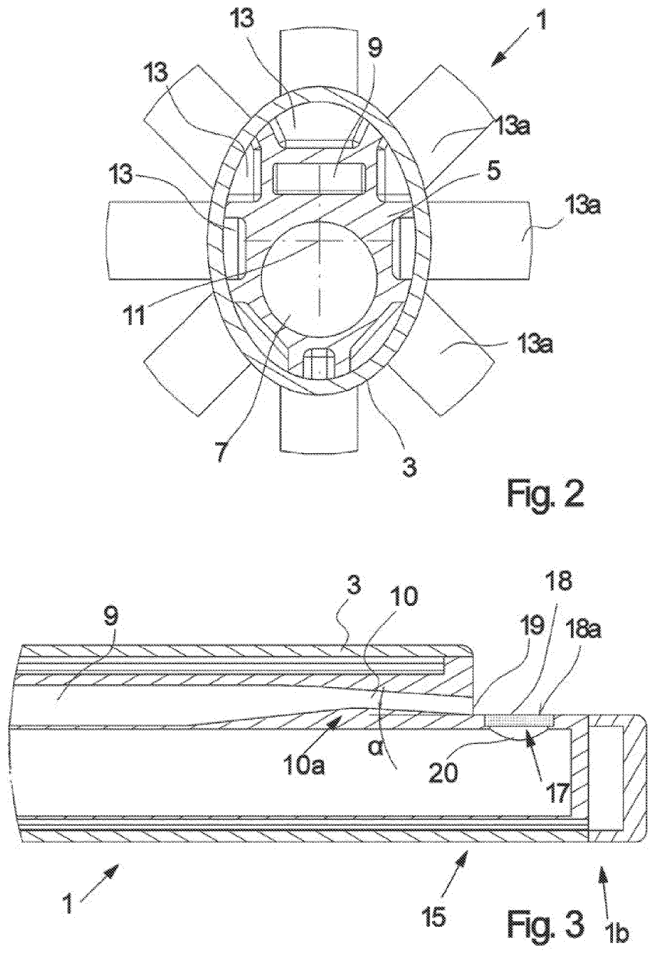

In FIGS. 1 and 2, a first embodiment of a probe housing 1 according to the invention is illustrated. The probe housing 1 is formed by a tubular outer body 3 and an inner body 5 arranged in the same. A sensor lead-through hole 7 and a flushing medium conduit 9 are arranged in the inner body 5. The flushing medium conduit 9 and the sensor lead-through hole 7 extend parallel to each other in the longitudinal direction of the probe housing 1 and are arranged offset from a center axis 11 of the probe housing 1. Recesses in the circumference of the inner body 5 form coolant conduits 13, the outer body 3 limiting the coolant conduits 13 in the radial direction. Further, at a first end 1a of the probe housing, ports 13a for the coolant conduits 13 are arranged circumferentially in a star shape.

The cross section of the coolant conduits 13 differ in size, whereby the cooling performance of the individual coolant conduits 13 is adapted to the thermal load on the probe housing 1. A coolant conduit 13 in a region of the probe housing 1 which in use faces a hot gas flow (top in FIG. 2 top), has a larger cross section than a coolant conduit in a region of the probe housing 1 which in use faces away from the hot gas flow (bottom in FIG. 2).

A sensor receptacle 15 is arranged at the end 1b opposite the first end 1a. A receiving space, not illustrated, for a sensor head is arranged in the sensor receptacle 15. The sensor receptacle 15 has a measuring section opening 17 communicating the receiving space with the environment. A sensor head received in the receiving space can perform a measurement through the measuring section opening 17.

The measuring section opening 17 comprises an optically transmissive disc 18 with a major surface 18a, the disc closing the measuring section opening 17. In the embodiment of the invention illustrated in FIGS. 1 and 2, the major surface 18a extends parallel to the center axis 11. In other words: Using the probe housing 1 illustrated in FIGS. 1 and 2, a measurement in a direction orthogonal to the center axis 11 is possible.

The tubular outer body 3 has an oval cross section. Thereby, the flow resistance of the probe housing 1 with respect to the hot gas flow may be reduced. The inner body 5 may be adapted to the shape of the outer body 3. Due to the oval cross section of the outer body 3, it is possible to arrange the flushing medium conduit 9 and the sensor lead-through hole 7 offset from the center axis 11 of the probe housing 1 in a particularly advantageous manner. Basically, the outer body 3 can also have a circular cross section.

The flushing medium conduit 9 comprises a flushing medium outlet line 10 terminating in a flushing medium outlet 19 arranged at the sensor receptacle 15. The flushing medium outlet 19 is arranged adjacent the measuring section opening 17 and is open to the measuring section opening 17. Thus, flushing medium flowing from the flushing medium outlet can generate a flow that flows over the major surface 18a of the disc 18. In this manner, a vortex formation of the flushing medium at the major surface 18a of the disc 18 and thus a tendency to contamination is avoided.

FIG. 3 shows a schematic sectional view of the probe housing in FIG. 1 with a first variant of the flushing medium conduit. The flushing medium outlet line 10 has a tapering cross section 10a for forming a nozzle shape. Due to the nozzle shape, it is possible to accelerate the flow of the flushing medium, whereby a particularly effective protection of the disc 18 against hot gas flow and a particularly good heat dissipation by the flushing medium.

The flushing medium outlet 19 is arranged under an acute angle α of 2° with respect to a plane in which the major surface 18a is located. Thereby, the flushing medium flows in a direction towards the major surface 18a. Due to the incident flow onto the major surface 18a of the disc 18, a particularly good thermal dissipation is achieved. In addition, the major surface 18a can be cleaned by the flushing medium flushing away particles adhering to the major surface.

The flushing medium outlet 19 has a rectangular cross section with a long side arranged parallel to the major surface 18a of the measuring section 17. In particular, it is provided that the flushing medium outlet 19 has a width in the direction of the long side, which is equal to or wider than the maximum dimension of the disc 18 in this direction. Thereby, it is ensured that the flushing medium can flush the entire disc 18.

The optically transmissive disc has an optical element 20 to collect optical radiation, e.g. a lens. This allows for enhanced measurements, since, in the case of an optically operating sensor, a larger amount of optical radiation is supplied to the sensor

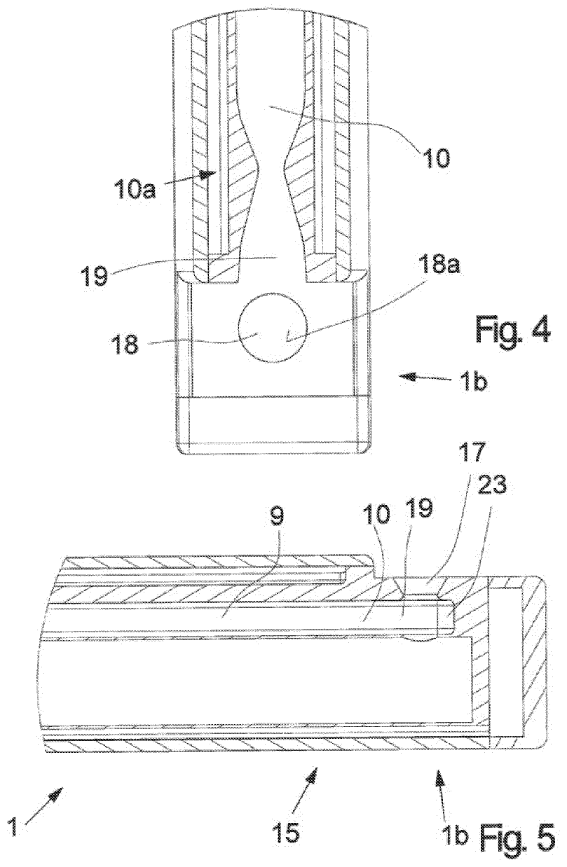

FIG. 4 is a schematic sectional view of the probe housing in FIG. 1 with a second variant of the flushing medium conduit.

The variant illustrated in FIG. 4 differs from the variant of the embodiment of FIG. 1 illustrated in FIG. 3 substantially by the shape of the flushing medium outlet line 10. In the variant illustrated in FIG. 4, the flushing medium outlet line 10 is designed as a Laval nozzle. Thereby, the flushing medium can be accelerated to very high velocities which are even higher than the velocity of sound. Thereby, it is possible to achieve a particularly effective protection against hot gas flow and a particularly good heat dissipation by the flushing medium. Here, the flushing medium outlet 19 can also be arranged parallel to the plane in which the major surface 18a is located, so that the flushing medium flows parallel to the major surface.

FIG. 5 is a schematic perspective view of a second embodiment of a probe housing 1 according to the invention for receiving a sensor. In contrast to the embodiment illustrated in FIG. 1, this embodiment has no optically transmissive disc arranged in the measuring section opening 17. The flushing medium outlet line 10 intersects the measuring section opening 17. The flushing medium flowing from the flushing medium outlet 19 flows transversely to the measuring section opening 17 and forms a sealing air flow which provides protection for a sensor head fitted in the senso receptacle 15 against the hot gas flow. In this embodiment, the flushing medium outlet line 10 can comprise a tapering cross section to form a nozzle form. However, an implementation without a tapering cross section is basically also possible.

The flushing medium conduit 9 comprises a flushing medium discharge section 23 arranged flush with the flushing medium outlet 19 on the side of the measuring section opening 17 opposite the flushing medium outlet 19. The flushing medium crossing the measuring section opening 17 is removed through the flushing medium discharge section 23 and a flushing medium return conduit not illustrated.

Due to the arrangement of the flushing medium conduit 9 and the sensor lead-through hole 7 parallel to each other and in the inner body 5, with the coolant conduits surround the flushing medium conduit 9 and the sensor lead-through hole 7, the probe housing 1 according to the invention can be designed to be very compact. Moreover, an improved cooling performance is achieved by the distributed arrangement of the coolant conduits 13. By providing the flushing medium outlet line 10 with a tapering cross section 10a for forming a nozzle shape, an advantageous flow of the flushing medium can be achieved in the region of the measuring section opening 17.

### LIST OF REFERENCE NUMERALS

1 probe housing
1a first end
1b opposite end
3 tubular outer body
5 inner body
7 sensor lead-through hole
9 flushing medium conduit
10 flushing medium outlet line
10a tapering cross section
11 center axis
13 coolant conduits
13a ports
15 sensor receptacle
17 measuring section opening
18 optically transmissive disc
18a major surface
19 flushing medium outlet
19a flushing medium port
20 optical element
23 flushing medium discharge section

1. A probe housing for accommodating sensors, comprising:
a plurality of coolant conduits distributed in the circumferential direction; at least one flushing medium conduit; a sensor lead-through hole, wherein the sensor lead-through hole extends at least partially parallel to the at least one flushing medium conduit; and a sensor receptacle into which the sensor lead-through hole opens, wherein the sensor receptacle has a measuring section opening, and the sensor receptacle has a flushing medium outlet conduit having a flushing medium outlet which is connected to the at least one flushing medium conduit,
wherein the flushing medium outlet conduit has a tapering cross-section to form a nozzle shape.

2. The probe housing according to claim 1, wherein an aperture and/or an optically transmissive disc is arranged in or at the measuring section opening, the disc having a major surface.

3. The probe housing according to claim 2, wherein the flushing medium outlet is arranged under an acute angle with respect to a plane in which the major surface is located.

4. The probe housing according to claim 1, wherein the flushing medium outlet line is designed as a Laval nozzle.

5. The probe housing according to claim 1, wherein the sensor lead-through hole and the at least one flushing medium conduit are surrounded by the coolant conduits and are arranged offset from a center axis of the probe housing.

6. The probe housing according to claim 2, wherein the flushing medium outlet has a rectangular cross section with a long side and a short side, and the flushing medium outlet is arranged with the long side parallel to the major surface of the aperture and/or the optically transmissive disc.

7. The probe housing according to claim 6, wherein the flushing medium outlet has a dimension in the direction of the long side that is equal or greater than the maximum dimension of the measuring section opening in that direction.

8. The probe housing according to claim 2, wherein the major surface of the aperture arranged in the measuring section opening and/or of the optically transmissive disc is arranged orthogonally or parallel to the center axis.

9. The probe housing according to claim 2, wherein the optically transmissive disc comprises an optical element.

10. The probe housing according to claim 1, wherein the flushing medium outlet line intersects the measuring section opening, a sealing air flow being creatable in the measuring section opening.

11. The probe housing according to claim 1, wherein a tubular outer body and an inner body surrounded by the outer body, the sensor lead-through hole and the at least one flushing medium conduit being are arranged in the inner body.

12. The probe housing according to claim 11, wherein the tubular outer body has an oval or elliptical cross section.

13. The probe housing according to claim 11, wherein the coolant conduits are arranged at the circumference of the inner body.

14. The probe housing according to claim 13, wherein coolant conduits are formed by recesses in the inner body, the outer body limiting the coolant conduits on at least one side.

15. A probe device with a sensor having a sensor head and a probe housing according to claim 1, the sensor head being arranged in the sensor receptacle and a measuring outlet of the sensor head is arranged facing to the measuring section opening.
